(19) United States
(12) Patent Application Publication
(10) Pub. No.: US 2023/0106669 A1
(43) Pub. Date: Apr. 6, 2023

(54) BINDING AFFINITY PREDICTION USING NEURAL NETWORKS

(71) Applicant: X Development LLC, Mountain View, CA (US)

(72) Inventors: Lance Ong-Siong Co Ting Keh, La Crescenta, CA (US); Ivan Grubisic, Oakland, CA (US); Ryan Jr. Poplin, Sunnyvale, CA (US); Ray Anthony Nagatani, JR., San Francisco, CA (US)

(21) Appl. No.: 17/486,407

(22) Filed: Sep. 27, 2021

(51) Int. Cl. G16B 15/10; G16B 40/20; G06N 3/04

(52) U.S. Cl. CPC G16B 15/10; G06N 3/0454; G16B 40/20

(57) ABSTRACT

Methods, systems, and apparatus, including computer programs encoded on a computer storage medium, for generating a binding prediction neural network. In one aspect, a method comprises: instantiating a plurality of structure prediction neural networks, wherein each structure prediction neural network has a respective neural network architecture and is configured to process data defining an input polynucleotide to generate data defining a predicted structure of the input polynucleotide; training each of the plurality of structure prediction neural networks; after training the plurality of structure prediction neural networks, determining a respective performance measure of each structure prediction neural network based at least in part on a prediction accuracy of the structure prediction neural network; and generating, based on the performance measures of the structure prediction neural networks, a binding prediction neural network.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a nonlinear transformation to a received input to generate an output.

### SUMMARY

This specification describes a transfer learning system and a polynucleotide optimization system implemented as computer programs on one or more computers in one or more locations.

According to a first aspect there is provided a method comprising: instantiating a plurality of structure prediction neural networks, wherein each structure prediction neural network has a respective neural network architecture and is configured to process data defining an input polynucleotide to generate data defining a predicted structure of the input polynucleotide; training each of the plurality of structure prediction neural networks on a set of structure prediction training data that comprises a plurality of training examples, wherein each training example comprises data defining: (i) a training polynucleotide, and (ii) a target structure of the training polynucleotide; after training the plurality of structure prediction neural networks, determining a respective performance measure of each structure prediction neural network based at least in part on a prediction accuracy of the structure prediction neural network; and generating, based on the performance measures of the structure prediction neural networks, a binding prediction neural network that is configured to process data defining an input polynucleotide to predict a binding affinity of the input polynucleotide for a specified binding target.

In some implementations, generating the binding prediction neural network based on the performance measures of the structure prediction neural networks comprises: identifying a best-performing structure prediction neural network from the plurality of structure prediction neural networks based on the performance measures; and generating the binding prediction neural network based on the best-performing structure prediction neural network.

In some implementations, identifying the best-performing structure prediction neural network from the plurality of structure prediction neural networks based on the performance measures comprises: identifying a structure prediction neural network associated with a highest performance measure from among the plurality of structure prediction neural networks as the best-performing structure prediction neural network.

In some implementations, the best-performing structure prediction neural network comprises an encoder subnetwork that is configured to process data defining an input polynucleotide to generate an embedded representation of the input polynucleotide, and generating the binding prediction neural network comprises: generating an encoder subnetwork of the binding prediction neural network that is configured to process an input polynucleotide to generate an embedded representation of the input polynucleotide, where a neural network architecture of the encoder subnetwork of the binding prediction neural network replicates a neural network architecture of the encoder subnetwork of the best-performing structure prediction neural network.

In some implementations, generating the encoder subnetwork of the binding prediction neural network comprises: initializing values of parameters of the encoder subnetwork of the binding prediction neural network based on trained values of parameters of the encoder subnetwork of the best-performing structure prediction neural network.

In some implementations, the method further comprises training the binding prediction neural network to perform a binding affinity prediction task, where the parameter values of the encoder subnetwork of the binding prediction neural network are not updated during the training of the binding prediction neural network.

In some implementations, the encoder subnetwork of the best-performing structure prediction neural network comprises a plurality of self-attention neural network layers.

In some implementations, for each of the plurality of structure prediction neural networks, determining the performance measure of the structure prediction neural network comprises: evaluating the prediction accuracy of the structure prediction neural network on a set of validation data.

In some implementations, for each training example in the structure prediction training data, the training polynucleotide is a ribonucleic acid (RNA).

In some implementations, for each training example in the structure prediction training data, the target structure of the training polynucleotide is a secondary structure of the training polynucleotide.

In some implementations, for each training example in the structure prediction training data, the target structure of the training polynucleotide is defined by a sequence of structure elements that each correspond to a respective nucleotide in the training polynucleotide.

In some implementations, the method further comprises training the binding prediction neural network on a set of binding prediction training data that comprises a plurality of training examples, where each training example comprises data defining: (i) a training polynucleotide, and (ii) a target binding affinity of the training polynucleotide for the specified binding target.

In some implementations, for each training example in the binding prediction training data, the training polynucleotide is a xeno nucleic acid (XNA).

In some implementations, for each training example in the binding prediction training data, the training polynucleotide is a threose nucleic acid (TNA).

In some implementations, the method further comprises using the binding prediction neural network to identify one or more polynucleotides as candidate polynucleotides that are predicted to bind to the specified binding target.

In some implementations, identifying one or more polynucleotides as candidate polynucleotides that are predicted to bind to the specified binding target comprises: using the binding prediction neural network to computationally evolve a population of polynucleotides over a plurality of evolutionary iterations; and after a last evolutionary iteration, identifying one or more polynucleotides from the population of polynucleotides as candidate polynucleotides.

In some implementations, the method further comprises: synthesizing the candidate polynucleotides; validating, using a high-throughput or low-throughput affinity assay, one or more of the candidate polynucleotides as being capable of binding to the specified binding target; and synthesizing a biologic using the one or more candidate polynucleotides validated as being capable of binding to the specified binding target.

In some implementations, the method further comprises administering the biologic to a subj ect.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Throughout this specification, a data element can refer to, e.g., a numerical value or an embedding. An embedding refers to an ordered collection of numerical values, e.g., a vector, matrix, or other tensor of numerical values.

The architecture of a neural network refers to the number of layers of the neural network, the operations performed by each of the layers (e.g., including the type of each of the layers), and the connectivity between the layers (e.g., which layers receive inputs from which other layers). Examples of possible types of neural network layers include fully-connected layers, attention layers, and convolutional layers.

A subnetwork refers to a neural network that is included in another, larger neural network.

A polynucleotide refers to a molecule that includes a sequence (chain) of chemically bonded nucleotides.

Each nucleotide is an organic molecule that includes: a phosphate, a backbone unit, and one of five standard nucleobases (in particular: adenine, guanine, cytosine, thymine, or uracil). The backbone unit can be, e.g., a ribose sugar (such that the sequence of nucleotides form a strand of ribonucleic acid (RNA)), a deoxyribose sugar (such that the sequence of nucleotides form a strand of deoxyribonucleic acid (DNA)), a substitute for ribose sugar and deoxyribose sugar (such that the sequence of nucleotides form a strand of xeno nucleic acid (XNA)), or combinations thereof.

Examples of substitutes for ribose sugar and deoxyribose sugar include: threose sugar (an XNA with threose sugar backbone units can be referred to as a threose nucleic acid (TNA)), glycol (an XNA with glycol backbone units can be referred to as a glycol nucleic acid (GNA)), and ribose that is modified to include a methylene bridge between the 2' oxygen and 4' carbon (an XNA with backbone units of modified ribose can be referred to as a locked nucleic acid (LNA)).

Data defining a polynucleotide can include a sequence of data elements that each identify the nucleobase included in a corresponding nucleotide in the sequence of nucleotides of the polynucleotide.

A structure of a polynucleotide generally characterizes a configuration of the nucleotides in the polynucleotide. For example, polynucleotide “secondary structure” refers to the structure induced from bonding (e.g., hydrogen bonding) of the nucleobases in the polynucleotide, e.g., to other nucleobases in the same polynucleotide or to nucleobases in other polynucleotides. As another example, polynucleotide “tertiary structure” refers to the structure induced from large-scale folding of the polynucleotide into a three-dimensional shape.

The structure of a polynucleotide can be represented by a sequence of “structure elements” (i.e., from a set of possible structure elements) that each correspond to a respective nucleotide in the polynucleotide. A structure element corresponding to a nucleotide characterizes the structure of the polynucleotide in the vicinity of the nucleotide. For example, for polynucleotide secondary structure, the set of possible structure elements can include: hairpin loops, internal loops, multi-branch loops, pseudoknots, dangling ends, and terminal mismatches. (Examples of possible secondary structure elements are illustrated with reference to FIG. 2). As another example, for polynucleotide tertiary structure, the set of possible structure elements can include: the type of helix (e.g., A-DNA, B-DNA or Z-DNA) and/or the number of helices (e.g., double helices, triple helices, and quadruple helices).

A binding affinity of a polynucleotide for a binding target generally measures a tendency of the polynucleotide to bind to the binding target. For example, a binding affinity of a polynucleotide for a binding target can be characterized by an association constant (or “binding constant”) Kₐ that measures a ratio of: the “on-rate constant” kₒₙ (which characterizes, at equilibrium, a quantity of the polynucleotide that is bound to the target) and the “off-rate constant” k_{off} (which characterizes, at equilibrium, a quantity of the polynucleotide that is not bound to the target). In this example, a higher binding affinity can indicate that a polynucleotide binds more strongly to a binding target.

Binding affinities of polynucleotides for a given binding target can be measured experimentally using, e.g., bio-layer interferometry, or systematic evolution of ligands by exponential enrichment (SELEX).

A binding affinity can be represented as a numerical value, e.g., a non-negative floating point numerical value.

A binding target for a polynucleotide can be, e.g., a protein, a protein complex, a peptide, a carbohydrate, an inorganic molecule, an organic molecule such as a metabolite, a cell, or any other appropriate target. A polynucleotide that binds to a target can be referred to as an “aptamer.”

Polynucleotides have been shown to selectively bind to specific targets with high binding affinity. Further, polynucleotides can be highly specific, in that a given polynucleotide may exhibit high binding affinity for one target but low binding affinity for many other targets. Thus, polynucleotides can be used to (for example) bind to disease-signature targets to facilitate a diagnostic process, bind to a treatment target to effectively deliver a treatment (e.g., a therapeutic or a cytotoxic agent linked to the polynucleotide), bind to target molecules within a mixture to facilitate purification, bind to a target to neutralize its biological effects, etc. However, the utility of a polynucleotide hinges largely on a degree to which it effectively binds to a target.

Frequently, an iterative experimental process (e.g., SELEX) is used to identify polynucleotides that selectively bind to target molecules with high affinity. In the iterative experimental process, a library of polynucleotides is incubated with a target molecule. Then, the target-bound polynucleotides are separated from the unbound polynucleotides and amplified via polymerase chain reaction (PCR) to seed a new pool of polynucleotides. This selection process is continued for a number (e.g., 6-15) of rounds with increasingly stringent conditions, which ensure that the polynucleotides obtained have the highest affinity to the target molecule.

The polynucleotide library typically includes 10¹⁴-10¹⁵ random polynucleotide sequences. However, there are approximately a septillion (10²⁴) different polynucleotides that could be considered. Exploring this full space of candidate polynucleotides is impractical. However, given that present-day experiments are now only a sliver of the full space, it is highly likely that optimal aptamer selection is not currently being achieved. This is particularly true when it is important to assess the degree to which polynucleotides bind with multiple different targets, as only a small portion of polynucleotides will have the desired combination of binding affinities across the targets. It would take an enormous amount of resources and time to experimentally evaluate a septillion (10²⁴) different polynucleotide sequences every time a new target is proposed.

The transfer learning system and the polynucleotide optimization system described in this specification provide a way of addressing this issue. In particular, given a binding target, the transfer learning system generates a binding prediction neural network that is configured to process data defining a polynucleotide to predict a binding affinity of the polynucleotide for the binding target. The polynucleotide optimization system uses the binding prediction neural network to computationally evolve a population of polynucleotides to identify one or more “candidate” polynucleotides that are predicted to have a high binding affinity for the binding target. The binding affinity of the candidate polynucleotides can be experimentally validated, and the candidate polynucleotides that are experimentally validated as having high binding affinity for the binding target can then synthesized for use as biologics, as will be described in more detail below.

Particular embodiments of the subject matter described in this specification can be implemented so as to realize one or more of the following advantages.

The transfer learning system and the polynucleotide optimization system described in this specification enable efficient identification of aptamers with high binding affinity for a binding target from a large space of possible polynucleotides (e.g., 10²⁴ polynucleotides). In particular, experiments are performed to evaluate the binding affinity of a proper subset of the space of possible nucleotides (e.g., 10¹⁴ polynucleotides out of 10²⁴ possible polynucleotides) for the binding target. The transfer learning system uses the experimentally measured binding affinities to train a binding prediction neural network that can predict the binding affinity of any polynucleotide for the binding target. The polynucleotide optimization system then uses the binding prediction neural network to computationally evolve a population of polynucleotides to identify one or more polynucleotides that are predicted to have a high binding affinity for the binding target. The transfer learning system and polynucleotide optimization system thus enable the space of possible nucleotides to be searched for aptamers for the binding target, while requiring experimental evaluation of the binding affinities for only a small subset of the space of possible polynucleotides.

Generally, high-throughput affinity assays (i.e., for evaluating binding affinities of polynucleotides for a binding target) can yield “noisy” binding affinity measurements, i.e., that include substantial inaccuracies. Low-throughput binding affinity assays can yield more accurate binding affinity measurements, but may not generate a large enough number of binding affinity measurements to enable training of a binding prediction neural network with a large number of parameters (e.g., with millions of parameters).

However, accurate structures (e.g., secondary structures) are known for large numbers of polynucleotides (e.g., RNAs). The transfer learning system leverages these large and accurate polynucleotide structure datasets to search a space of neural network architectures to identify a structure prediction neural network that can effectively predict polynucleotide structures. The transfer learning system then reuses part of the architecture (and optionally, the parameter values) of the structure prediction neural network to instantiate and train a binding prediction neural network for predicting polynucleotide binding affinities.

The task of predicting polynucleotide structure is related to the task of predicting polynucleotide binding affinity, e.g., because the binding affinity of a polynucleotide for a target is partially a function of the structure of the polynucleotide.

Moreover, predicting polynucleotide structures is a “sequence-to-sequence” prediction task and thus provides a rich training signal for adapting the parameters of a structure prediction neural network to generate effective internal representations of polynucleotides, e.g., as compared to the “sequence-to-scalar” prediction task of predicting binding affinities. In particular, performing the sequence-to-sequence task of predicting polynucleotide structure requires the structure prediction neural network to generate an internal representation of an input polynucleotide that encodes enough information to enable the generation of a complex sequence of structure elements that characterize each nucleotide in the input polynucleotide.

Therefore, generating the binding prediction neural network using the architecture (and, optionally, the parameter values) of a structure prediction neural network can enable the binding prediction neural network achieve a higher prediction accuracy while being trained over fewer training iterations and using less training data. Training the binding prediction neural network over fewer training iterations and using less training data reduces consumption of computational resources, e.g., memory and computing power.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example transfer learning system and an example polynucleotide optimization system.

FIG. 2 shows an example transfer learning system.

FIGS. 3A - 3D illustrate examples of polynucleotide secondary structures.

FIG. 4 shows an example architecture of a structure prediction neural network.

FIG. 5 shows an example architecture of a binding prediction neural network.

FIG. 6 shows an example polynucleotide optimization system.

FIG. 7 is a flow diagram of an example process for generating a binding prediction neural network.

Like reference numbers and designations in the various drawings indicate like elements.

### DETAILED DESCRIPTION

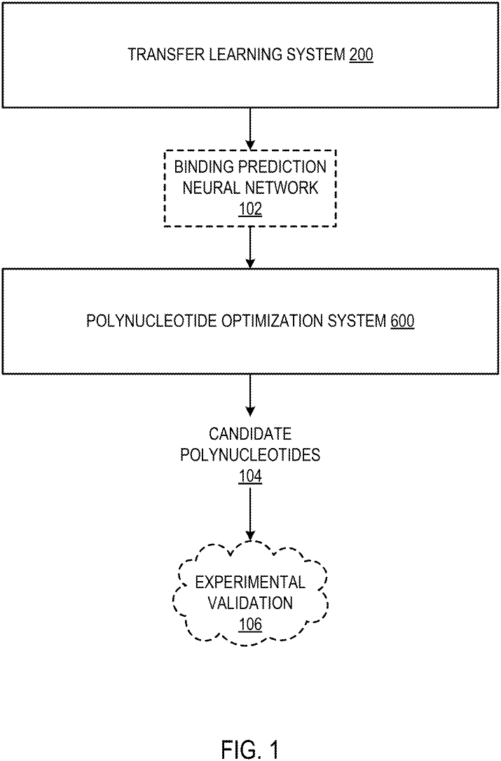

FIG. 1 shows an example transfer learning system 200 and an example polynucleotide optimization system 600.

The transfer learning system 200 and the polynucleotide optimization system 600 are used to identify polynucleotides (aptamers) that are predicted to have a high binding affinity for a binding target (e.g., which can be specified by a user). The binding target can be, e.g., a protein, a protein complex, a peptide, a carbohydrate, an inorganic molecule, an organic molecule such as a metabolite, or a cell. The identified polynucleotides can be used (for example) to bind to disease-signature targets to facilitate a diagnostic process, to bind to a treatment target to effectively deliver a treatment (e.g., a therapeutic or a cytotoxic agent linked to the polynucleotide), to bind to target molecules within a mixture to facilitate purification, or to bind to a target to neutralize its biological effects.

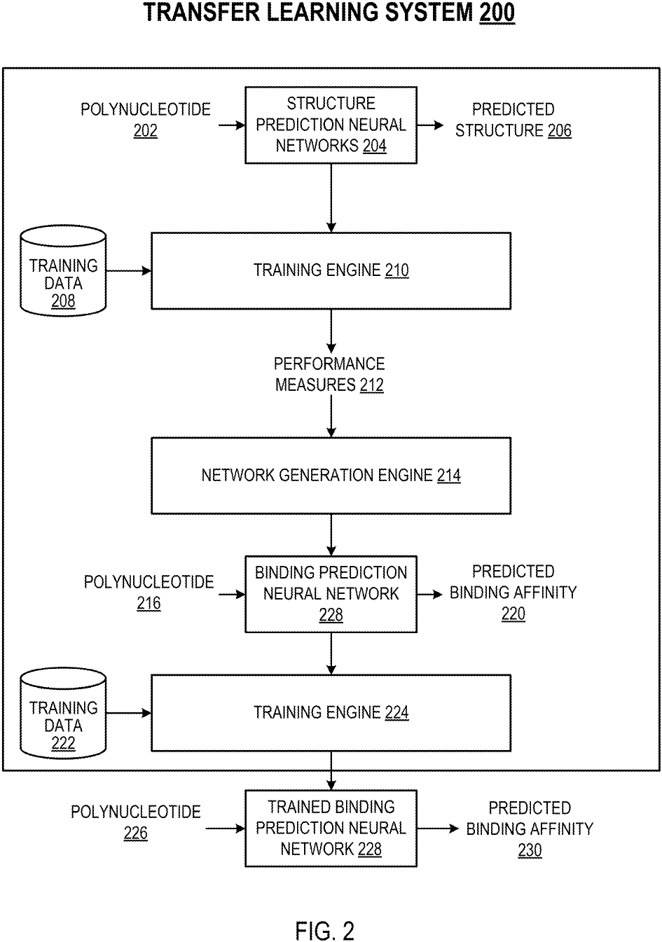

The transfer learning system 200, which is described in more detail with reference to FIG. 2, generates a binding prediction neural network 102 that is configured to process data defining a polynucleotide to predict a binding affinity of the polynucleotide for a binding target.

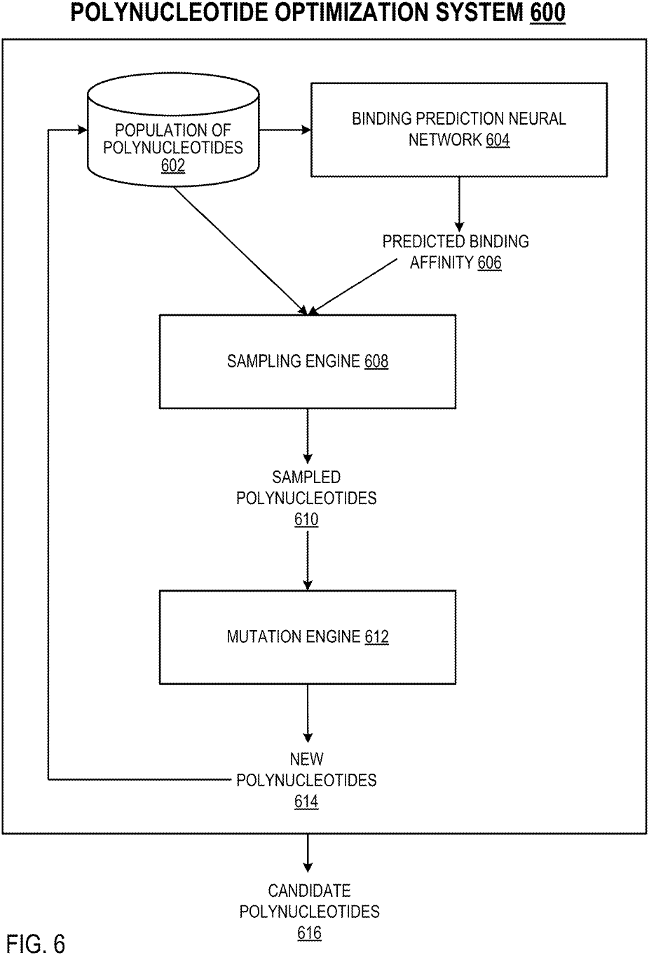

The polynucleotide optimization system 600, which is described in more detail with reference to FIG. 6, uses the binding prediction neural network 102 to computationally evolve a population of polynucleotides to identify one or more “candidate” polynucleotides that are predicted to have a high binding affinity for the binding target.

The candidate polynucleotides 104 can be physically synthesized, and their binding affinity for the binding target can be experimentally validated 106, e.g., using a high-throughput affinity assay such as a binding selection assay (e.g., phage display) or a low-throughput affinity assay such as bio-layer interferometry.

A biologic can be synthesized using one or more of the polynucleotides that are experimentally validated as having a high binding affinity for the binding target. (The binding affinity of a polynucleotide for a binding target can be referred to as being “high,” e.g., if it satisfies a predefined threshold). The biologic may be used as a new drug, a therapeutic tool, a diagnostic tool, a drug delivery device, or for any other appropriate purpose. In particular, the biologic can be used as part of a treatment that is administered to a subject.

In some implementations, the candidate polynucleotides 104 generated using the transfer learning system 200 and the polynucleotide optimization system are XNA aptamers, e.g., TNA aptamers. XNA aptamers may be particularly well suited for use as biologics because, unlike DNA and RNA aptamers, they are not readily recognized and degraded by nucleases in the body.

FIG. 2 shows an example transfer learning system 200. The transfer learning system 200 is an example of a system implemented as computer programs on one or more computers in one or more locations in which the systems, components, and techniques described below are implemented.

The system 200 generates a binding prediction neural network 228 that is configured to process data defining a polynucleotide 226 to generate a predicted binding affinity 230 of the polynucleotide 226 for a binding target. Data defining a polynucleotide 226 can include, e.g., a sequence of data elements that each identify the nucleobase included in a respective nucleotide in the sequence of nucleotides of the polynucleotide 226, as described above.

To generate the binding prediction neural network 228, the system 200 initially instantiates a set of structure prediction neural networks 204 that each have a respective neural network architecture. (Example techniques for selecting the architectures of the structure prediction neural networks 204 are described below).

Each structure prediction neural network 204 is configured to process data defining a polynucleotide 202 to generate data defining a predicted structure 206 of the polynucleotide 202. More specifically, each structure prediction neural network 204 is configured to process data defining a polynucleotide 202 to generate, for each nucleotide in the polynucleotide, a respective score distribution over a set of possible structure elements. The structure prediction neural network then selects a respective structure element for each nucleotide based on the corresponding score distribution over the set of possible structure elements. For example, the structure prediction neural network can select a respective structure element for each nucleotide as the possible structure element having the highest score under the corresponding score distribution.

After instantiating a structure prediction neural network 204, the system 200 uses a training engine 210 to determine a performance measure 212 of the structure prediction neural network 204. To determine a performance measure 212 of the structure prediction neural network 204, the training engine 210 trains the structure prediction neural network 204 on a set of training data 208, and then evaluates its performance on a set of validation data, as will be described in more detail next.

The training data 208 includes multiple training examples, where each training example includes data defining: (i) a polynucleotide, and (ii) a “target” (i.e., actual) structure of the polynucleotide. The target structure of the polynucleotide can be represented, e.g., as a sequence of “target” structure elements that each correspond to a respective nucleotide in the polynucleotide, and that collectively define the structure of the polynucleotide. The target polynucleotide structures in the training data may have been determined using physical experiments, e.g., x-ray crystallography or nuclear magnetic resonance (NMR) imaging.

The training engine 210 can train the structure prediction neural network 204 on the training data 208 over multiple training iterations.

Prior to the first training iteration, the training engine 210 can initialize the parameter values of the structure prediction neural network 204 using any appropriate neural network parameter initialization technique, e.g., random initialization, where the value of each parameter is sampled from a predefined probability distribution, glorot initialization, and so on. Subsequently, at each training iteration, the training engine 210 can sample a “batch” (set) of training examples from the training data 208, and train the structure prediction neural network 204 on each training example in the batch.

To train the structure prediction neural network 204 on a training example, the training engine 210 processes data defining the polynucleotide specified by the training example using the structure prediction neural network 204 to generate, for each nucleotide, a respective score distribution over the set of possible structure elements. The training engine 210 can then determine gradients of an objective function that, for each nucleotide, measures an error between: (i) the score distribution over the set of possible structure elements generated by the structure prediction neural network for the nucleotide, and (ii) the target structure element for the nucleotide. The objective function can measure the error for each nucleotide, e.g., as a cross-entropy entropy error. The training engine 210 can then update the parameter values of the structure prediction neural network 204 using the gradients of the objective function for the batch of training examples.

The training engine 210 can determine gradients of the objective function with respect to the parameters of the structure prediction neural network using, e.g., backpropagation. The training engine 210 can update the parameter values of the structure prediction neural network based on gradients of the objective function using any appropriate gradient descent optimization algorithm, e.g., RMSprop or Adam.

After training the structure prediction neural network 204 on the training data 208 (e.g., for a predefined number of training iterations), the training engine 210 determines a performance measure 212 of the structure prediction neural network 204. The performance measure 212 of the structure prediction neural network 204 measures the prediction accuracy of the structure prediction neural network 204 on a set of validation data.

The validation data, like the training data 208, includes multiple training examples, where each training example includes data defining: (i) a polynucleotide, and (ii) a target structure of the polynucleotide. The validation data is generally “held out” from the training of the structure prediction neural network 204, i.e., the training engine 210 does not train the structure prediction neural network 204 on the training examples in the validation data.

The training engine 210 can measure the prediction accuracy of the structure prediction neural network 204 on the validation data in any appropriate way. For example, the training engine 210 can evaluate the prediction accuracy of the structure prediction neural network 204 for each training example in the validation data. The training engine 210 can then determine the performance measure 212 of the structure prediction neural network 204 as the average prediction accuracy of the structure prediction neural network 204 across the training examples of the validation data.

To evaluate the prediction accuracy of the structure prediction neural network 204 for a training example in the validation data, the training engine 210 can provide data defining the polynucleotide specified by the training example as an input to the structure prediction neural network 204. The structure prediction neural network 204 can process the data defining the polynucleotide to generate a respective score distribution over the set of possible structure elements for each nucleotide in the polynucleotide. The training engine 210 can then determine the prediction accuracy by evaluating an objective function based on the score distributions generated by the structure prediction neural network 204 and the target structure specified by the training example, as described above. The objective function used to evaluate the prediction accuracy of the structure prediction neural network 204 for the training example in the validation data can optionally be different than the objective function used during training of the structure prediction neural network 204. (In some cases, a lower value of the objective function can indicate a higher prediction accuracy).

Each structure prediction neural network 204 has a neural network architecture from a set of possible structure prediction neural network architectures. Each possible structure prediction neural network architecture includes: (i) a respective “encoder” subnetwork, and (ii) a respective “decoder” subnetwork. The encoder subnetwork of a structure prediction neural network is configured to process data defining a polynucleotide to generate an embedded representation of the polynucleotide. The decoder subnetwork of a structure prediction neural network is configured to process an embedded representation of a polynucleotide to generate data defining a predicted structure of the polynucleotide.

The set of possible structure prediction neural network architectures is parameterized by a set of hyper-parameters. That is, each possible set of hyper-parameter values (i.e., that includes a respective value for each hyper-parameter in the set of hyper-parameters) specifies a respective architecture in the set of possible structure prediction neural network architectures. In particular, each possible set of hyper-parameter values can specify the number, type, and configuration of the neural network layers in a structure prediction neural network architecture.

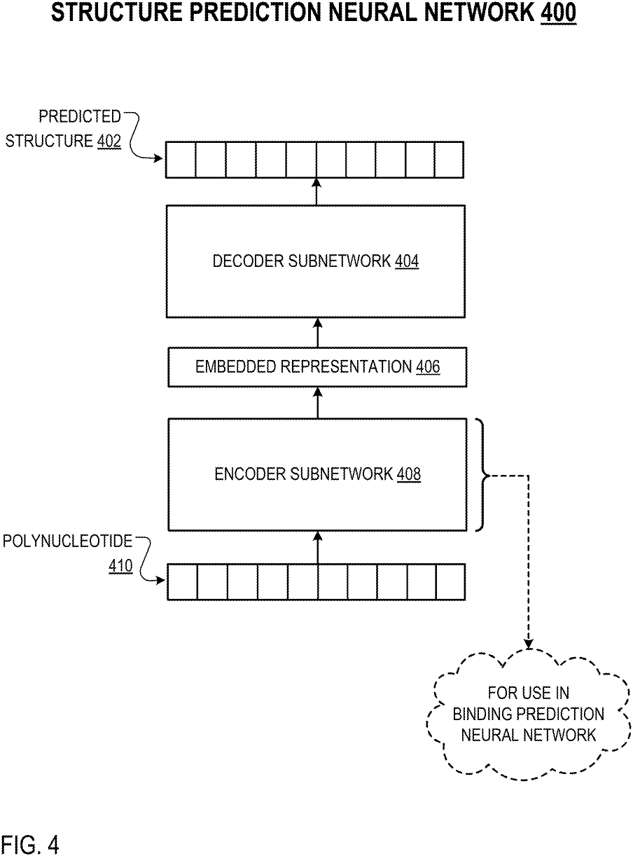

Examples of structure prediction neural network architectures, and of hyper-parameters parametrizing a set of possible structure prediction neural network architectures, are described in more detail with reference to FIG. 4.

Optionally, the set of hyper-parameters parameterizing the set of possible structure prediction neural network architectures can include both: (i) a set of “architectural” hyper-parameters, and (ii) a set of “training” hyper-parameters. The set of architectural hyper-parameters can specify a possible neural network architecture, as described above. The set of training hyper-parameters can include hyper-parameters of a training algorithm to be used by the training engine 210 for training a structure prediction neural network having the neural network architecture specified by the architectural hyper-parameters.

The set of training hyper-parameters can include, e.g., a learning rate hyper-parameter, a dropout rate hyper-parameter, a hyper-parameter that scales a regularization term in the objective function, a batch size hyper-parameter, an optimizer hyper-parameter, a training duration hyper-parameter, or any other appropriate training algorithm hyper-parameters. A learning rate hyper-parameter can specify a scaling factor to be applied to gradients of an objective function prior to the gradients being used to update the values of structure prediction neural network parameters during training. A dropout rate hyper-parameter can specify a probability of dropping (i.e., removing) neurons from the structure prediction neural network during training, e.g., as part of regularizing the training of the structure prediction neural network. A batch size hyper-parameter can specify a number of training examples included in each batch during training of structure prediction neural network parameters by stochastic gradient descent. An optimizer hyper-parameter can specify the optimizer used to update structure prediction neural network parameters during training, e.g., RMSprop or Adam. A training duration hyper-parameter can specify a number of training iterations (e.g., of stochastic gradient descent) to be performed during training of structure prediction neural network parameters.

To instantiate each structure prediction neural network 204, the system 200 selects values of the hyper-parameters parametrizing the set of possible structure prediction neural network architectures. The system 200 then generates a structure prediction neural network 204 having the architecture specified by the selected hyper-parameter values. If the set of hyper-parameters include training hyper-parameters, as described above, then training engine 210 trains the structure prediction neural network 204 in accordance with the selected values of the training hyper-parameters.

The system 200 can select the respective hyper-parameter values specifying the architecture of each structure prediction neural network 204 in any of a variety of possible ways. A few example techniques for selecting hyper-parameter values specifying structure prediction neural network architectures are described next.

In some implementations, to select hyper-parameter values specifying a structure prediction neural network architecture, the system 200 randomly selects a respective value of each hyper-parameter in the set of hyper-parameters.

In some implementations, the system 200 selects hyper-parameter values specifying structure prediction neural network architectures using an optimization technique. More specifically, each structure prediction neural network architecture can be associated with a respective performance measure 212 that characterizes a performance of the architecture on a polynucleotide structure prediction task, as described above. The system 200 can thus select hyper-parameter values to optimize the performance measures 212 of the corresponding structure prediction neural network architectures.

For example, the system 200 can initialize values of the set of hyper-parameters that parameterize the set of possible structure prediction neural network architectures, e.g., by randomly initializing the hyper-parameter values. At each iteration in a sequence of iterations, the system 200 can determine a performance measure 212 of a structure prediction neural network architecture specified by current values of the set of hyper-parameters. The system can use an appropriate optimization technique to update the current values of the set of hyper-parameters to encourage an increase in the performance measures 212 of structure prediction neural network architectures generated at subsequent iterations. That is, in this example, the system 200 can generate a sequence of structural prediction neural networks, where the architecture of each structure prediction neural network is determined based on the performance measures of previously generated structure prediction neural networks.

The optimization technique can be, e.g., a black-box optimization technique, e.g., as described with reference to Golovin, D., Solnik, B., Moitra, S., Kochanski, G., Karro, J., & Sculley, D.: “Google vizier: A service for black-box optimization,” In Proceedings of the 23rd ACM SIGKDD International Conference on Knowledge Discovery and Data Mining, pp. 1487-1495 (2017). As another example, the optimization technique can be, e.g., a reinforcement learning optimization technique, e.g., as described with reference to Zoph, B., Le, Q.V.: “Neural architecture search with reinforcement learning,” arXiv: 1611.01578v2 (2017).

In some implementations, the hyper-parameter values specifying the respective architectures of one or more of the structure prediction neural networks 204 can be provided to the system 200 by a user, e.g., through an application programming interface (API) of the system 200.

After the system 200 determines the performance measures 212 for the structure prediction neural networks 204, a network generation engine 214 generates a binding prediction neural network 228 based on the performance measures 212.

The binding prediction neural network 228 is configured to process data defining a polynucleotide 216 to generate a predicted binding affinity 220 of the polynucleotide for the binding target. The binding prediction neural network 228 has a neural network architecture that includes: (i) an “encoder” subnetwork, and (ii) a “regression” subnetwork. The encoder subnetwork of the binding prediction neural network is configured to process data defining a polynucleotide to generate an embedded representation of the polynucleotide. The regression subnetwork of the binding prediction neural network is configured to process an embedded representation of a polynucleotide to generate data defining a predicted binding affinity of the polynucleotide for the binding target.

To generate the binding prediction neural network 228, a structure prediction neural network 204 is identified as being a “best-performing” structure prediction neural network based on the performance measures 212. For example, a structure prediction neural network 204 having the best (e.g., highest) performance measure 212 (i.e., from among the structure prediction neural networks 204) can be identified as the best-performing structure prediction neural network.

The network generation engine 214 generates the encoder subnetwork of the binding prediction neural network 228 with the same neural network architecture as the encoder subnetwork of the best-performing structure prediction neural network. That is, the architecture of the encoder subnetwork of the binding prediction neural network replicates the architecture of the encoder subnetwork of the best-performing structure prediction neural network.

Optionally, the network generation engine 214 can initialize the parameter values of the encoder subnetwork of the binding prediction neural network 228 with the trained parameter values of the encoder subnetwork of the best-performing structure prediction neural network.

Thus the network generation engine 214 reuses the architecture, and optionally, the trained parameter values, of the encoder subnetwork of the best-performing structure prediction neural network as the encoder subnetwork of the binding prediction neural network 228.

The network generation engine 214 can generate the regression subnetwork of the binding prediction neural network 228 with any appropriate neural network architecture that enables it to perform its described function, i.e., processing an embedded representation of a polynucleotide to generate a predicted binding affinity. The network generation engine 214 can initialize the parameter values of the regression subnetwork of the binding prediction neural network 228 in any appropriate manner, e.g., the network generation engine 214 can randomly sample a respective value for each parameter of the regression subnetwork from a predefined probability distribution.

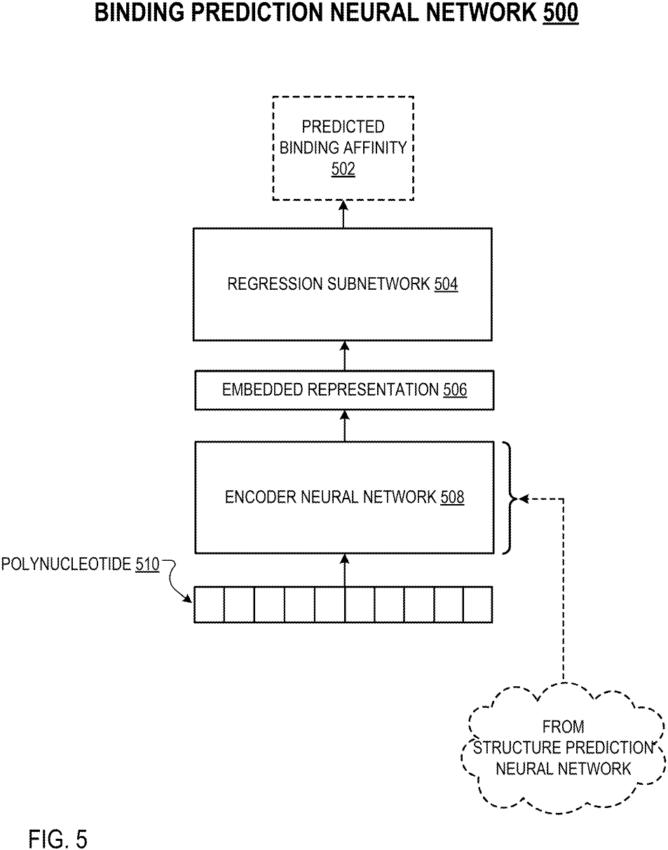

An example architecture of a binding prediction neural network 228 is described in more detail with reference to FIG. 5.

After generating the binding prediction neural network 228, the system 200 uses a training engine 224 to train the binding prediction neural network 228 on a set of training data 222. Optionally, if the hyper-parameters specifying the architecture of the best-performing structure prediction neural network include training hyper-parameters, then the training engine 224 trains the binding prediction neural network 228 in accordance with those training hyper-parameters. That is, the training engine 224 can reuse the training hyper-parameters that were used to train the best-performing structure prediction neural network in the training of the binding prediction neural network 228.

The training data 222 includes multiple training examples, where each training example includes data defining: (i) a polynucleotide, and (ii) a “target” (i.e., actual) binding affinity of the polynucleotide for the binding target. The target binding affinities of the training data 222 can be generated using experimental techniques, e.g., bio-layer interferometry or SELEX.

The training engine 224 can train the binding prediction neural network 228 on the training data 222 over multiple training iterations. At each training iteration, the training engine 224 can sample a “batch” (set) of training examples from the training data 222, and train the binding prediction neural network 228 on each training example in the batch.

To train the binding prediction neural network 228 on a training example, the training engine 224 processes data defining the polynucleotide specified by the training example using the binding prediction neural network 228 to generate a predicted binding affinity of the polynucleotide for the binding target. The training engine 224 can then determine gradients of an objective function that measures an error between: (i) the predicted binding affinity generated by the binding prediction neural network, and (ii) the target binding affinity. The objective function can measure the error between the predicted and target binding affinities, e.g., as a squared error. The training engine 224 can then update the parameter values of the binding prediction neural network 228 using gradients of the objective function with respect to the parameters of the binding prediction neural network.

In some implementations, prior to training the binding prediction neural network 228, the system 200 initializes the parameters of the encoder subnetwork of the binding prediction neural network 228 with the trained parameter values of the encoder subnetwork of the best-performing structure prediction neural network, as described above.

In these implementations, the training engine 224 can optionally “freeze” the parameter values of the encoder subnetwork, i.e., by refraining from updating the parameter values of the encoder subnetwork during training. That is, the training engine 224 optionally trains the parameter values of only the regression subnetwork of the binding prediction neural network 228, while treating the parameter values of the encoder subnetwork as static values. Freezing the parameter values of the encoder subnetwork can accelerate the training of the binding prediction neural network 228, e.g., by reducing the number of parameters that require training. Freezing the parameter values of the encoder subnetwork can also reduce the likelihood of the binding prediction neural network 228 overfitting the training data 222. Moreover, as a result of being trained on the polynucleotide structure prediction task, the parameters of the encoder subnetwork can generate effective embedded representations of polynucleotides even without being trained on the binding affinity prediction task.

As an alternative to freezing the parameter values of the encoder subnetwork of the binding prediction neural network 228, the training engine 224 can train the parameters of the encoder subnetwork using a lower learning rate while training the parameters of the decoder subnetwork using a higher learning rate. The learning rate for a parameter refers to a scaling factor applied to a gradient of the objective function with respect to the parameter prior to the gradient being used to update the value of the parameter. Training the parameters of the encoder subnetwork using the lower learning rate allows them to be gradually adapted to the binding affinity prediction task, thus increasing the prediction accuracy of the binding prediction neural network 228.

After being trained, the binding prediction neural network 228 can be provided for use by the polynucleotide optimization system described with reference to FIG. 6.

In some implementations, the structure prediction neural networks 204 are trained on training data 208 characterizing RNA structures, and the binding prediction neural network 228 is trained on training data 222 characterizing TNA aptamer binding affinities.

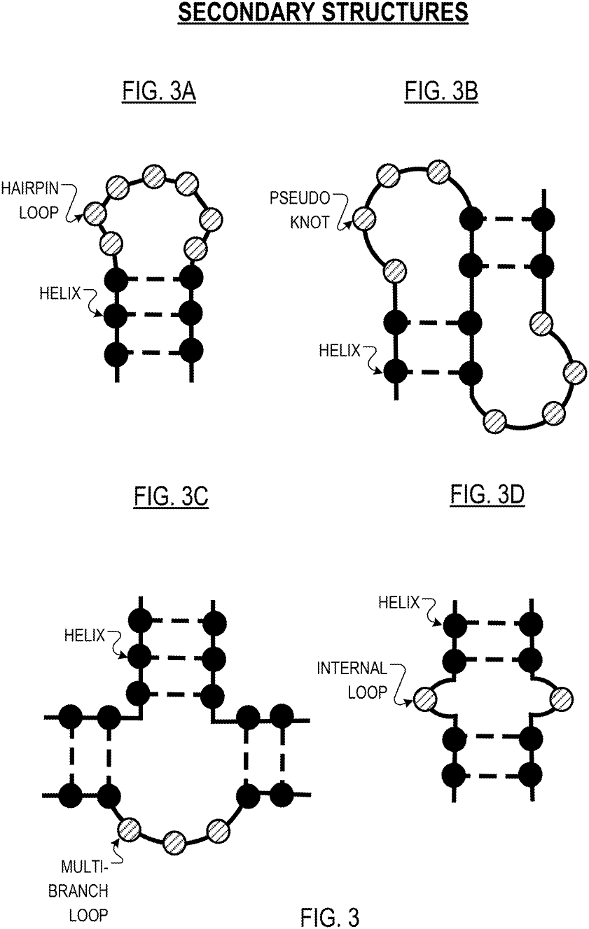

FIG. 3A - FIG. 3D illustrate examples of polynucleotide secondary structures. The circles represent nucleotides, the solid lines represent covalent bonds between nucleotides, and the broken lines represent bonds (e.g., hydrogen bonds) between nucleobases.

In FIG. 3A, the nucleotides represented as dark circles have a “helix” secondary structure, and the nucleotides represented as hatched circles have a “hairpin loop” secondary structure.

In FIG. 3B, the nucleotides represented as dark circles have a “helix” secondary structure, and the nucleotides represented as hatched circles have a “pseudoknot” secondary structure.

In FIG. 3C, the nucleotides represented as dark circles have a “helix” secondary structure, and the nucleotides represented as hatched circles have a “multi-branch loop” secondary structure.

In FIG. 3D, the nucleotides represented as dark circles have a “helix” secondary structure, and the nucleotides represented as hatched circles have an “internal loop” secondary structure.

FIG. 4 shows an example architecture of a structure prediction neural network 400. The structure prediction neural network 400 is configured to process data defining a polynucleotide 410 to generate data defining a predicted structure 402 of the polynucleotide 410.

The structure prediction neural network 400 includes an encoder subnetwork 408 and a decoder subnetwork 404.

The encoder subnetwork 408 is configured to process data defining the polynucleotide 410 to generate an embedded representation 406 of the polynucleotide 410. The architecture, and optionally, the trained parameter values, of the encoder subnetwork of the best-performing structure prediction neural network can be reused as the encoder subnetwork of the binding prediction neural network.

The decoder subnetwork 404 is configured to process the embedded representation 406 of the polynucleotide 410 to generate data defining the predicted structure 402 of the polynucleotide 410.

The encoder subnetwork 408 and the decoder subnetwork 404 can have any appropriate neural network architectures which enable them to perform their described functions. In particular, the encoder subnetwork 408 and the decoder subnetwork 404 can include any appropriate types of neural network layers (e.g., fully-connected layers, convolutional layers, attention layers, etc.) in any appropriate numbers (e.g., 5 layers, 10 layers, or 25 layers) and connected in any appropriate configuration (e.g., as a linear sequence of layers).

Example architectures of the encoder subnetwork 408 and the decoder subnetwork 404 are described next.

In some implementations, the encoder subnetwork 408 includes an embedding layer followed by a sequence of one or more encoder “stacks” (i.e., where a stack refers to a set of neural network layers).

The embedding layer of the encoder subnetwork is configured to receive data defining the polynucleotide 410, in particular, a sequence of data elements that each identify the nucleobase included in a corresponding nucleotide in the polynucleotide 410. The embedding layer maps the data defining the polynucleotide 410 to a collection of embeddings that includes a respective embedding corresponding to each position in the sequence of nucleotides of polynucleotide 410. The embedding corresponding to a position in the sequence of nucleotides of the polynucleotide 410 can be, e.g., a one-hot embedding identifying the nucleobase included in the nucleotide at the position. Optionally, for each position in the sequence of nucleotides of the polynucleotide 410, the embedding layer can combine (e.g., sum or average) the embedding for the position with a positional embedding representing an index of the position.

Each encoder stack of the encoder subnetwork is configured to receive a set of input embeddings (including a respective embedding corresponding to each position in the sequence of nucleotides of the polynucleotide 410), and update each input embedding to generate a corresponding set of updated embeddings. The first encoder stack receives the embeddings generated by the embedding layer, and each subsequent encoder stack receives the embeddings generated by the preceding encoder stack. The updated embeddings generated by the final encoder stack collectively define the embedded representation 406 of the polynucleotide 410.

Each encoder stack can include a sequence of one or more self-attention neural network layers, e.g., that are configured to receive a set of input embeddings and to update the input embeddings by a self-attention operation, e.g., a query-key-value self-attention operation. Optionally, the self-attention operation can be a “multi-head” self-attention operation, i.e., where each “head” implements a respective self-attention operation parameterized by a respective set of parameters, and the self-attention layer combines (e.g., averages) the updated embeddings from each head to generate the output embeddings of the self-attention layer. Each encoder stack can further include one or more fully-connected neural network layers that process the embeddings generated by the final self-attention layer of the encoder stack to generate updated embeddings.

In some implementations, the decoder subnetwork 404 is configured to autoregressively generate a sequence of structure elements defining the predicted structure 402 of the polynucleotide 410. More specifically, the decoder subnetwork generates a respective structure element corresponding to each nucleotide in the sequence of nucleotides of the polynucleotide 410 in order, starting from the first nucleotide in the sequence. To generate the structure element for a given nucleotide in the sequence of nucleotides, the decoder subnetwork 404 processes: (i) the embedded representation of the polynucleotide 410, and (ii) data defining respective structure elements for any preceding nucleotides in the sequence of nucleotides.

The decoder subnetwork 404 can include an embedding layer followed by a sequence of decoder stacks and an output layer. For convenience, the embedding layer, the decoder stacks, and the output layer of the decoder subnetwork are described in the following with reference to the operations performed to generate a structure element for a “current” nucleotide in the sequence of nucleotides of the polynucleotide 410.

The embedding layer of the decoder subnetwork is configured to receive data identifying a respective structure element for each nucleotide that precedes the current nucleotide in the sequence of nucleotides. If the current nucleotide is the first nucleotide in the sequence of nucleotides (i.e., such that there are no preceding nucleotides), the embedding layer can generate a predefined embedding. Otherwise, if the current nucleotide is after the first nucleotide in the sequence of nucleotides, the embedding layer generates a collection of embeddings that includes a respective embedding corresponding to each nucleotide that precedes the current nucleotide. The embedding corresponding to a nucleotide can be, e.g., a one-hot embedding identifying the structure element previously generated by the structure prediction neural network 400 for the nucleotide. Optionally, for each nucleotide that precedes the current nucleotide, the embedding layer can combine (e.g., sum or average) the embedding for the nucleotide with a positional embedding representing the position of the nucleotide in the sequence of nucleotides.

Each decoder stack of the decoder subnetwork is configured to receive: (i) a set of input embeddings representing the nucleotides that precede the current nucleotide, and (ii) the embedded representation 406 of the polynucleotide 410, and to update each input embedding to generate a corresponding set of updated embeddings. The first decoder stack receives the embeddings generated by the embedding layer of the decoder subnetwork, and each subsequent decoder stack receives the embeddings generated by the preceding decoder stack. The updated embeddings generated by the final decoder stack are provided to the output layer of the decoder subnetwork.

Each decoder stack can include a sequence of attention neural network layers, including one or more self-attention neural network layers and one or more cross-attention neural network layers. Each self-attention neural network layer can be configured to receive a set of input embeddings and to update the input embeddings by a self-attention operation, e.g., a query-key-value self-attention operation. Optionally, the self-attention operation can be a multi-head self-attention operation. Each cross-attention neural network layer can be configured to receive a set of input embeddings and to update the input embeddings by an attention operation over the collection of embeddings that collectively define the embedded representation of the polynucleotide 410. Optionally, the cross-attention operation can be a multi-head cross-attention operation. Each decoder stack can further include one or more fully-connected neural network layers that process the embeddings generated by the final attention layer of the decoder stack to generate updated embeddings.

The output layer of the decoder subnetwork 404 is configured to process the updated embeddings generated by the final decoder stack to generate a score distribution over a set of possible structure elements. The structure prediction neural network can select a structure element for the current nucleotide in accordance with the score distribution, e.g., by selecting the possible structure element having the highest score as structure element for the current nucleotide.

It can be appreciated that the example structure prediction neural network architecture described above can be parameterized by a set of hyper-parameters, e.g., hyper-parameters specifying: the number of encoder stacks, the number of decoder stacks, the number of heads in the self-attention neural network layers of the encoder stacks, the number of heads in the self-attention neural network layers of the decoder stacks, the number of heads in the cross-attention neural network layers of the decoder stacks, the number of fully-connected layers in each encoder stack, the number of fully-connected layers in each decoder stack, the parameterization of the positional embeddings used by the embedding layer of the encoder subnetwork, the parametrization of the positional embeddings using by the embedding layer of the decoder subnetwork, the dimensionality of the embeddings generated by the embedding layer of the encoder subnetwork, the dimensionality of the embeddings generated by the embedding layer of the decoder subnetwork, and epsilon hyper-parameters of layer normalization operations performed by the encoder and decoder subnetworks. Thus this set of hyper-parameters can be understood as parametrizing a set of possible structure prediction neural network architectures.

FIG. 5 shows an example architecture of a binding prediction neural network 500. The binding prediction neural network 500 is configured to process data defining a polynucleotide 510 to generate data defining a predicted binding affinity 502 of the polynucleotide 510 for a binding target.

The binding prediction neural network 500 includes an encoder subnetwork 508 and a regression subnetwork 504.

The encoder subnetwork 508 is configured to process data defining the polynucleotide 510 to generate an embedded representation 506 of the polynucleotide 510. The architecture of the encoder subnetwork 508 of the binding prediction neural network 500 can replicate the architecture of the encoder subnetwork of the (best-performing) structure prediction neural network. Example architectures of the encoder subnetwork of the structure prediction neural network are described above with reference to FIG. 4.

The regression subnetwork 504 is configured to process the embedded representation 506 of the polynucleotide 510 to generate the predicted binding affinity 502 of the polynucleotide 510 for the binding target.

The regression subnetwork 504 can have any appropriate neural network architecture that enables it to perform its described functions. For example, as described with reference to FIG. 4, the embedded representation 506 can include a collection of embeddings. The regression subnetwork 504 can process the embeddings by a pooling layer (e.g., an average pooling layer) to generate a combined embedding, and process the combined embedding by a fully-connected layer to generate the predicted binding affinity 502.

FIG. 6 shows an example polynucleotide optimization system 600. The polynucleotide optimization system 600 is an example of a system implemented as computer programs on one or more computers in one or more locations in which the systems, components, and techniques described below are implemented.

The system 600 is configured to computationally evolve a population (i.e., set) of polynucleotides 602 using a binding prediction neural network 604 to generate a set of candidate polynucleotides 616 that are predicted to have a high binding affinity for a binding target. The binding prediction neural network 604 can be generated, e.g., by the transfer learning system 200 described with reference to FIG. 2.

The system 600 can initialize the population of polynucleotides 602 in any appropriate way. A few example techniques for initializing the population 602 are described next.

In one example, the system 600 can initialize the population 602 with a set of randomly generated polynucleotides. The system 600 can randomly generate a polynucleotide, e.g., by randomly selecting the length of the polynucleotide, and by randomly selecting the identity of the nucleobase included in each nucleotide of the polynucleotide. (The length of a polynucleotide refers to the number of nucleotides in the polynucleotide).

As another example, to initialize the population 602, the system 600 can obtain an input polynucleotide that is known (e.g., from prior experiments) to bind to the binding target. The system 600 can then process the input polynucleotide to generate a set of additional polynucleotides. In particular, the system 600 can generate each additional polynucleotide by applying one or more random “mutations” (i.e., modifications) to the input polynucleotide. For example, to generate an additional polynucleotide, the system 600 can randomly select one or more nucleotides in the input polynucleotide. Then, for each selected nucleotide, the system 600 can modify the nucleotide to include a nucleobase that is randomly selected from the set of possible nucleobases.

After initializing the population 602, the system 600 processes each polynucleotide in the population 602 using the binding prediction neural network 604 to generate a respective predicted binding affinity 606 that predicts the binding affinity of the polynucleotide for the binding target.

The system 600 uses a sampling engine 608 and a mutation engine 612 to update the population 602 with one or more new polynucleotides 614 at each of multiple evolutionary iterations.

More specifically, at each evolutionary iteration, the sampling engine 608 samples one or more polynucleotides 610 from the population 602 in accordance with the predicted binding affinities 606 of the polynucleotides in population 602. Generally, the sampling engine 608 samples polynucleotides from the population 602 such that polynucleotides associated with higher predicted binding affinities have a higher likelihood of being sampled. For example, the sampling engine 608 can process the predicted binding affinities associated with the polynucleotides in the population 602, e.g., using a soft-max function, to generate a probability distribution over the polynucleotides in the population 602. The sampling engine 608 can then sample one or more polynucleotides 610 from the population 602 in accordance with the probability distribution.

Next, the mutation engine 612 processes the sampled polynucleotides 610 to generate one or more new polynucleotides 614. A few example techniques by which the mutation engine 612 can generate the new polynucleotides 614 from the sampled polynucleotides are described next.

In one example, the mutation engine 612 can generate one or more new polynucleotides 614 from each sampled polynucleotide 610 by applying one or more random mutations to the sampled polynucleotide 610 (as described above).

In another example, the mutation engine 612 generates each new polynucleotide 614 as a combination of two sampled polynucleotides 610. For example, to generate a new polynucleotide 614 from two sampled polynucleotides (including a “first” and “second” sampled polynucleotide), the mutation engine 612 can sample respective “crossover” positions in the nucleotide sequences of the first and second sampled polynucleotides. (Generally, the crossover position in a nucleotide sequence is neither the first position nor the last position in the nucleotide sequence). The mutation engine 612 then replaces the subsequence of the first polynucleotide that follows the crossover position in the first polynucleotide by the corresponding subsequence of the second polynucleotide that follows the crossover position in the second polynucleotide.

The system 600 processes each new polynucleotide 614 using the binding prediction neural network 604 to generate a corresponding predicted binding affinity 606, adds the new polynucleotides 614 to the population 602, and proceeds to the next evolutionary iteration.

The system 600 thus evolves the population 602 over the sequence of evolutionary iterations, where polynucleotides having higher predicted binding affinities are more likely to be selected for “reproduction,” i.e., to be used to generate new polynucleotides 614 to be added to the population 602.

After determining that a termination criterion is satisfied, the system 600 can identify one or more polynucleotides from the population 602 as candidate polynucleotides 616, and output the candidate polynucleotides 616. The termination criterion may be, e.g., that the system has completed a predefined number of evolutionary iterations. The system 600 can identify a polynucleotide from the population 602 as a candidate polynucleotide based on the predicted binding affinity 606 of the polynucleotide. For example, the system 600 can identify a polynucleotide from the population 602 as a candidate polynucleotide 616 if the predicted binding affinity of the polynucleotide satisfies a predefined threshold.

The candidate polynucleotides 616 can be physically synthesized, and their binding affinity for the binding target can be experimentally validated, as described above. A biologic can then be synthesized using one or more of the polynucleotides that are experimentally validated as having a high binding affinity for the binding target.

Optionally, the experimentally validated binding affinities of the candidate polynucleotides 616 can be used to generate new training examples for training the binding prediction neural network 604, and the binding prediction neural network 604 can be trained on the new training examples. After the binding prediction neural network 604 is trained on the new training examples, the system 600 can repeat the process of computationally evolving a population of polynucleotides 602 using the binding prediction neural network 604 to generate additional candidate polynucleotides 616.

FIG. 6 provides one example implementation of a polynucleotide optimization system that can use a binding prediction neural network to generate one or more polynucleotides that are predicted to have a high binding affinity for a binding target. Other implementations of the polynucleotide optimization system are possible. That is, there are a variety of possible ways that a polynucleotide optimization system can use a binding prediction neural network to identify polynucleotides that are predicted to have a high binding affinity for a binding target.

For example, in another implementation, the polynucleotide optimization system can iteratively adjust a polynucleotide over a sequence of optimization iterations to generate a polynucleotide that is predicted to have a high binding affinity for a binding target.

More specifically, prior to the first optimization iteration, the polynucleotide optimization system can initialize a “current” polynucleotide, e.g., by randomly generating the current polynucleotide, or by obtaining a current polynucleotide that is known (e.g., from prior experiments) to bind to the binding target.

At each optimization iteration, the polynucleotide optimization system can update the current polynucleotide based on a predicted binding affinity of the current polynucleotide for the binding target. More specifically, the polynucleotide optimization system can process the current polynucleotide using the binding prediction neural network to generate a predicted binding affinity of the current polynucleotide for the binding target. As part of processing the current polynucleotide, an embedding layer of the binding prediction neural network can generate a respective embedding (e.g., one-hot embedding) of each nucleotide in the polynucleotide, e.g., that identifies the nucleobase included in the polynucleotide. The polynucleotide optimization system can determine respective gradients of the predicted binding affinity with respect to the nucleotide embeddings representing the current polynucleotide, e.g., by backpropagation. The polynucleotide optimization system can use the gradients to update the nucleotide embeddings, e.g., using any appropriate gradient ascent optimization procedure. The polynucleotide optimization system can then update the current polynucleotide based on the updated nucleotide embeddings.

The polynucleotide optimization system can update the current polynucleotide based on the updated nucleotide embeddings in a variety of possible ways. For example, for each nucleotide in the polynucleotide, the polynucleotide optimization system can process the corresponding updated nucleotide embedding, e.g., using a soft-max function, to generate a probability distribution over the set of possible nucleobases. The polynucleotide optimization system can then sample a nucleobase in accordance with the probability distribution, and update the nucleotide to be a nucleotide that includes the sampled nucleobase.

Thus, over a sequence of optimization iterations, the polynucleotide optimization system can iteratively update the current polynucleotide by gradient ascent to optimize the predicted binding affinity of the current polynucleotide for the binding target.

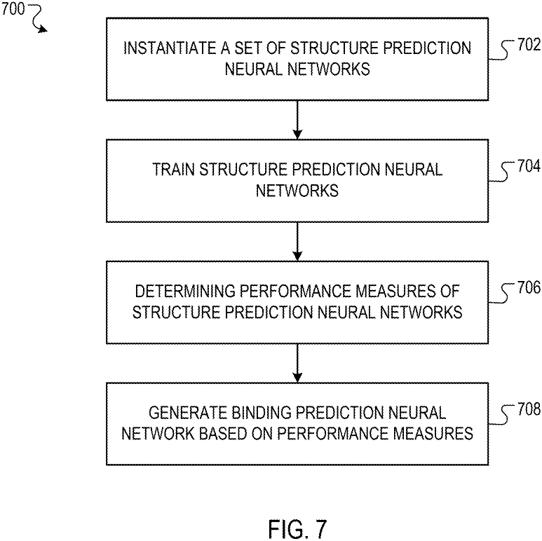

FIG. 7 is a flow diagram of an example process 700 for generating a binding prediction neural network. For convenience, the process 700 will be described as being performed by a system of one or more computers located in one or more locations. For example, a transfer learning system, e.g., the transfer learning system 200 of FIG. 2, appropriately programmed in accordance with this specification, can perform the process 700.

The system instantiates a set of structure prediction neural networks (702). Each structure prediction neural network has a respective neural network architecture and is configured to process data defining an input polynucleotide to generate data defining a predicted structure of the input polynucleotide.

The system trains each structure prediction neural network on a set of structure prediction training data (704). The training data includes a set of training examples, where each training example includes data defining: (i) a training polynucleotide, and (ii) a target structure of the training polynucleotide.

After training the structure prediction neural networks, the system determines a respective performance measure of each structure prediction neural network based at least in part on a prediction accuracy of the structure prediction neural network (706).

The system generates a binding prediction neural network based on the performance measures of the structure prediction neural networks (708). The binding prediction neural network is configured to process data defining an input polynucleotide to predict a binding affinity of the input polynucleotide for a specified binding target.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user’s device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
instantiating a plurality of structure prediction neural networks, wherein each structure prediction neural network has a respective neural network architecture and is configured to process data defining an input polynucleotide to generate data defining a predicted structure of the input polynucleotide;
training each of the plurality of structure prediction neural networks on a set of structure prediction training data that comprises a plurality of training examples, wherein each training example comprises data defining: (i) a training polynucleotide, and (ii) a target structure of the training polynucleotide;
after training the plurality of structure prediction neural networks, determining a respective performance measure of each structure prediction neural network based at least in part on a prediction accuracy of the structure prediction neural network; and
generating, based on the performance measures of the structure prediction neural networks, a binding prediction neural network that is configured to process data defining an input polynucleotide to predict a binding affinity of the input polynucleotide for a specified binding target.

2. The method of claim 1, wherein generating the binding prediction neural network based on the performance measures of the structure prediction neural networks comprises:
identifying a best-performing structure prediction neural network from the plurality of structure prediction neural networks based on the performance measures; and
generating the binding prediction neural network based on the best-performing structure prediction neural network.

3. The method of claim 2, wherein identifying the best-performing structure prediction neural network from the plurality of structure prediction neural networks based on the performance measures comprises:
identifying a structure prediction neural network associated with a highest performance measure from among the plurality of structure prediction neural networks as the best-performing structure prediction neural network.

4. The method of claim 2, wherein the best-performing structure prediction neural network comprises an encoder subnetwork that is configured to process data defining an input polynucleotide to generate an embedded representation of the input polynucleotide, and wherein generating the binding prediction neural network comprises:
generating an encoder subnetwork of the binding prediction neural network that is configured to process an input polynucleotide to generate an embedded representation of the input polynucleotide,
wherein a neural network architecture of the encoder subnetwork of the binding prediction neural network replicates a neural network architecture of the encoder subnetwork of the best-performing structure prediction neural network.

5. The method of claim 4, wherein generating the encoder subnetwork of the binding prediction neural network comprises:
initializing values of parameters of the encoder subnetwork of the binding prediction neural network based on trained values of parameters of the encoder subnetwork of the best-performing structure prediction neural network.

6. The method of claim 5, further comprising training the binding prediction neural network to perform a binding affinity prediction task, wherein the parameter values of the encoder subnetwork of the binding prediction neural network are not updated during the training of the binding prediction neural network.

7. The method of claim 4, wherein the encoder subnetwork of the best-performing structure prediction neural network comprises a plurality of self-attention neural network layers.

8. The method of claim 1, wherein for each of the plurality of structure prediction neural networks, determining the performance measure of the structure prediction neural network comprises:
evaluating the prediction accuracy of the structure prediction neural network on a set of validation data.

9. The method of claim 1, wherein for each training example in the structure prediction training data, the training polynucleotide is a ribonucleic acid (RNA).

10. The method of claim 1, wherein for each training example in the structure prediction training data, the target structure of the training polynucleotide is a secondary structure of the training polynucleotide.

11. The method of claim 1, wherein for each training example in the structure prediction training data, the target structure of the training polynucleotide is defined by a sequence of structure elements that each correspond to a respective nucleotide in the training polynucleotide.

12. The method of claim 1, further comprising training the binding prediction neural network on a set of binding prediction training data that comprises a plurality of training examples, wherein each training example comprises data defining: (i) a training polynucleotide, and (ii) a target binding affinity of the training polynucleotide for the specified binding target.

13. The method of claim 12, wherein for each training example in the binding prediction training data, the training polynucleotide is a xeno nucleic acid (XNA).

14. The method of claim 13, wherein for each training example in the binding prediction training data, the training polynucleotide is a threose nucleic acid (TNA).

15. The method of claim 1, further comprising using the binding prediction neural network to identify one or more polynucleotides as candidate polynucleotides that are predicted to bind to the specified binding target.

16. The method of claim 15, wherein identifying one or more polynucleotides as candidate polynucleotides that are predicted to bind to the specified binding target comprises:
using the binding prediction neural network to computationally evolve a population of polynucleotides over a plurality of evolutionary iterations; and
after a last evolutionary iteration, identifying one or more polynucleotides from the population of polynucleotides as candidate polynucleotides.

17. The method of claim 15, further comprising:
synthesizing the candidate polynucleotides;
validating, using a high-throughput or low-throughput affinity assay, one or more of the candidate polynucleotides as being capable of binding to the specified binding target; and
synthesizing a biologic using the one or more candidate polynucleotides validated as being capable of binding to the specified binding target.

18. The method of claim 17, further comprising administering the biologic to a subject.

19. A system comprising:
one or more computers; and
one or more storage devices communicatively coupled to the one or more computers, wherein the one or more storage devices store instructions that, when executed by the one or more computers, cause the one or more computers to perform operations comprising:
instantiating a plurality of structure prediction neural networks, wherein each structure prediction neural network has a respective neural network architecture and is configured to process data defining an input polynucleotide to generate data defining a predicted structure of the input polynucleotide;
training each of the plurality of structure prediction neural networks on a set of structure prediction training data that comprises a plurality of training examples, wherein each training example comprises data defining: (i) a training polynucleotide, and (ii) a target structure of the training polynucleotide;
after training the plurality of structure prediction neural networks, determining a respective performance measure of each structure prediction neural network based at least in part on a prediction accuracy of the structure prediction neural network; and
generating, based on the performance measures of the structure prediction neural networks, a binding prediction neural network that is configured to process data defining an input polynucleotide to predict a binding affinity of the input polynucleotide for a specified binding target.

20. One or more non-transitory computer storage media storing instructions that when executed by one or more computers cause the one or more computers to perform operations comprising:
instantiating a plurality of structure prediction neural networks, wherein each structure prediction neural network has a respective neural network architecture and is configured to process data defining an input polynucleotide to generate data defining a predicted structure of the input polynucleotide;
training each of the plurality of structure prediction neural networks on a set of structure prediction training data that comprises a plurality of training examples, wherein each training example comprises data defining: (i) a training polynucleotide, and (ii) a target structure of the training polynucleotide;
after training the plurality of structure prediction neural networks, determining a respective performance measure of each structure prediction neural network based at least in part on a prediction accuracy of the structure prediction neural network; and
generating, based on the performance measures of the structure prediction neural networks, a binding prediction neural network that is configured to process data defining an input polynucleotide to predict a binding affinity of the input polynucleotide for a specified binding target.
